(19) United States
(12) Patent Application Publication
(10) Pub. No.: US 2021/0147785 A1
(43) Pub. Date: May 20, 2021

(54) PORTABLE INCUBATION DEVICE

(71) Applicant: BIOAVLEE SP. Z O. O, Wroclaw (PL)

(72) Inventors: Igor BUZALEWICZ, Wroclaw (PL); Marek JAROSLAWSKI, Bystrzyca (PL); Damian ANDRZEJEWSKI, Sinzing (DE)

(21) Appl. No.: 16/624,271

(22) PCT Filed: Nov. 9, 2017
(86) PCT No.: PCT/PL2017/050055

§ 371 Date: Dec. 18, 2019

(30) Foreign Application Priority Data
Jun. 19, 2017 (PL) P.421928

(51) Int. Cl. C12M 1/00; C12M 1/22; C12M 1/36

(52) U.S. Cl. CPC C12M 41/14; C12M 41/48; C12M 23/10

(57) ABSTRACT

The invention relates to portable incubation device comprising a device housing (14) accommodating an incubation chamber housing (3) defining an incubation chamber (9) with an opening (4) together with a feed system (1) and a temperature control system of the heating module (2), characterised in that in the incubation chamber (9) there is at least one heating system (6) comprising at least one heating module (7), preferably two, wherein on the heating system (6) there is a supported a frame (10) supporting a culturing dish (11), and wherein the temperature control system of the heating module (2) provides continuous real time regulation of the desired incubation temperature in the incubation chamber (9).

The invention relates to a portable incubator device for customised culture of microorganisms on liquid and solid nutritive substrates located in culture dished. The invention is applicable in microbiology.

Laboratory incubators are devices for culturing prokaryotic and eukaryotic cells at a predetermined temperature that maintain conditions favourable for their growth in liquid and solid culture media. Even minor deviations in the temperature and composition of the atmosphere inside the incubation chamber, compared to optimal conditions, can have an adverse effect on the proliferation rate of cell cultures. The description of the U.S. Pat. No. 6,060,266, “Self-contained incubator for growth of microorganism” discloses an invention generally relating to the field of detection and deactivation of microorganisms. In particular, the invention relates to the use of an incubator for the detection of microorganisms and the use of storage tanks for the sterilisation of microorganisms. The described incubation device is designed to facilitate the growth of the microorganism in liquid samples, also during transport. It also provides the possibility to carry out microbiological tests on site without the need to send samples to the laboratory. The device does not use any electrical components or subunits. The use of the liquid sample container and the container cap provides easy disinfection of the sample and secure transport thereof. By separating the sample from the disinfectant by means of a membrane or a septum, the contact between the sample and the biocide is impossible until the user breaks or removes the septum or membrane, at which point the disinfection of the sample becomes possible. Said device for detecting and culturing microorganism comprises the following: a storage container for microorganism samples, a heating unit that can be actuated to deliver heat to said sample container, at least one thermal shield disposed between the sample container and the heating unit; an insulating container accommodating the sample container and the heating unit sufficiently close to each other so as to maintain the appropriate incubation temperature inside the said sample container. This device is adapted to provide the appropriate incubation temperature for the growth of microorganisms. The device is dedicated for liquid samples. The heating unit means a chemical source of heat that may contain the following elements: iron particles, activated charcoal particles, celluloses, zeolite. Such heating units can be activated in order to produce heat without the need for using additional special electrical components. Generally, they can be activated by shaking. It is also possible to use as the heating unit a gel that is able to maintain and discharge heat over several hours. Such gels, however, require an external source of thermal energy for activation. The heating units used should allow to maintain the heat of 10 or 100 ml of fluid in the chamber at a temperature above 300° C. and 350° C., and below approximately 40° C. or 45° C. for approximately 20 to 30 hours. Heating units are placed on both sides of the sample. This device enables mobile incubation of individual different microbiological samples in liquid form, without the possibility, however, of obtaining a distribution of the incubation temperature that would be uniform in time and ensuring an incubation of samples constant over time. The description of the patent EP 0182 926 B1 entitled “Self-sufficient incubation assembly” discloses a self-sufficient incubation device consisting of a basic part made of a suitable thermal insulator, and a cover. In the basic part there is a deeply embedded dish for cell culture, a set containing a heating element readily connectable to an electric power source, and cells with nutrient agar can be placed in a suitable rigid thermoplastic fastening. In a particularly advantageous embodiment, the self-sufficient incubation system is provided with an additional optical system allowing the recording of enlarged images of the microorganism colonies. The incubation chamber containing the fixation for the culture dish on the bottom is separated by a thermally conductive plate from the heating element to obtain a temperature in the range of 35.8° C. -36.2° C. One of the variants of the said invention is the use of electro-optical methods for measuring the intensity of light passing through cells located in a liquid medium in an optically transparent cuvette, by introducing a suitable light source and a detector which, in a given period of time, are characterised by bacterial growth or proliferation by measuring changes in the optical density of the liquid medium. In this case, bacterial cells are in liquid medium and in a transparent cuvette. As in the previous case, it is possible to incubate in a mobile manner all biological samples only at a constant and strictly predetermined temperature. It is not possible to incubate different samples at different temperatures. The description of the patent U.S. Pat. No. 2,124,250 A entitled “Bacteria plate cabinet” discloses an invention which provides useful improvements relative to preparation procedures of bacterial samples and incubation thereof under secure conditions until laboratory observations. The device has a box structure with a vertical septum dividing the inside of the box structure into a chamber for biological samples stacked on a support, a heating chamber and a boiler arranged above the heating chamber. The water in the boiler is heated by the lamp so that the samples, after they are collected, are very quickly dried on the plates. After drying the samples, the plates that contain them are removed and placed on the support in the chamber for biological samples until laboratory testing. The device enables the drying of microbiological samples and their storage, without, however, allowing for ensuring individual temperature conditions to incubate different samples present in the chamber for biological samples. The description of the patent U.S. Pat. No. 3,553,426 A entitled. “Temperature control apparatus” discloses an invention in the form of a device for controlling the temperature. The objective of said invention is to provide an incubation device which allows for achieving a constant temperature covering the total incubation area within the incubation chamber. In addition, said devices enables simultaneous incubation of cells in an environment with different gas compositions, but at a predetermined temperature. The device for controlling the cell incubation temperature includes a housing accommodated in a heating element that substantially surrounds the entire incubation area. The interior of the device can be divided into separate chambers, allowing for an independent change of atmosphere and introduction of gases without affecting the atmosphere of the other chambers or other chambers. An additional feature is the use of a thermostat designed to stabilise and maintain a constant overall temperature inside the device. All biological samples present in the device can be incubated at the same constant temperature, but it is impossible to incubate different samples at different temperatures in the subsequent incubation chambers. The description of the patent U.S. Pat. No. 7,838,286 B2 entitled “Incubator” discloses an invention that relates to an incubator which can prevent air from penetrating into the incubation chamber from the outside of the incubator by blocking the entry of the external air from the rear side of the incubator to its front side. According to said invention, the incubator comprises a main body of an adiabatic box with an opening on its front surface, an adiabatic door mounted to the main body of the adiabatic box, an open/closed transparent interior door, an incubation chamber surrounded by the internal door and the main body of the adiabatic box, in which samples can be incubated such as cells or microorganisms. The main body of the adiabatic box contains metallic external boxes, a metal internal box, thermal insulation material placed in the external box between the external box and the internal box, and a space filled with air. The pressure inside the incubation chamber exceeds that in the space external to the incubator. The incubator allows for obtaining a constant temperature distribution inside the incubation chamber, which makes it possible to culture multiple samples at the same constant temperature, without, however, allowing for culturing multiple samples at different temperatures at the same time. The description of the patent US 2014/0030802 A1 entitled “Conditioning chamber for storing samples in a time-controlled manner and method for storing samples in a time-controlled manner” discloses an invention for providing a controlled space for storing samples in a time-controlled manner, comprising a device for automatically feeding containers with samples to a closed air-conditioned space having at least one wall, wherein the container for samples can be inserted into the space of the sealed chamber through an opening, and wherein the feed device has at least one drive and control unit and an arm of the incubator for receiving the samples is arranged inside the sealed air-conditioned space.

The invention is characterised by the fact that the device has an automatic feeding arm that grips the container with a sample from the receiving position and deposits the container with a sample in the chamber in the deposition position in the incubator. As in previous cases, incubation of all the samples present in the air-conditioned chamber of the incubator takes place at constant temperature, without the possibility of incubating separate samples at different temperatures. The description of the patent EP 2 821 477 B1 entitled “Cell culture device having culture medium replacement function” discloses a technical solution proposing a device for culturing small samples of bacterial and fungal cells with the function of replacing the culture medium in a small number of small Petri dishes. Said invention is capable of performing an automatic replacement of the culture medium and cell culture in the same receptacle, which greatly reduces the cost of the apparatus. The device comprises a drive chamber, an incubation chamber located under the drive chamber, a rotatable shaft body and a motorized rotatable shaft body, wherein at least one set of top and bottom disks is horizontally attached to the rotatable shaft body within the incubator chamber, the Petri dishes containing cells and culture mediums are arranged on the bottom disc, and their caps are arranged on the top disc so that it is possible to achieve a state in which the bottom and top discs are approximated one to the other in a vertical direction so as to cover the Petri dish with the cap, and condition, in which the bottom and top discs are vertically separated from each other, and one of the bottom discs is rotated by a predetermined angle to obtain a lateral displacement of the Petri dish and the cap thereof, so that the Petri dishes are partially exposed, with the replacement of the culture medium taking place at the exposed portion of the Petri dish, from the top of the opening located on the top disc, and after the medium is replaced, the bottom and top discs are rotated and moved vertically until the Petri dish is covered with the top cover in order to continue the culture of cells. The incubator chamber may be filled with the desired gas mixture and allow for incubating all samples at a suitable constant temperature and humidity. However, it is not possible to simultaneously incubate biological samples in the chamber at different temperatures. The description of the patent EP 3 103 864 A1 discloses an invention relating in particular to an incubator of Petri microdishes, but without limitation, to the incubation of embryonic samples as a part of in vitro fertilisation (IVF) procedures. The invention further relates to a method for maintaining thermal equilibrium in cell culture samples, and to an optical method for testing cell culture samples in an incubator. The aim of the present invention is to provide a rentable incubator in which samples of incubated cells can be cultured at a constant temperature, while being observable by means of a standard stationary laboratory microscope. Said invention includes a concept of an incubator for regulating the temperature of cell culture samples, which incubator comprises: a support element having a base portion that supports cell culture samples; and a cap that covers the support element; wherein the base part of the support element is at least partially thermally conductive at or near the sample, and it comprises a surface heating system consisting of: a heating element, a temperature equalising layer arranged between and being in communication with the heating element and the base part. The heating element is a printed circuit board which comprises suitably formed paths and may further comprise an electrically insulating layer arranged between the heating element and the temperature equalising layer. The container for cell culture sample can consist of cylindrical culture wells in which Petri microdishes are placed. By using the element/material dissipating thermal energy from the heating element within the incubator and using the temperature equalising layer, it is possible to prevent significant temperature differences occurring between adjacent Petri microdishes which could lead to uneven temperature of sample incubation. The developed solution allows for incubating microorganisms cultured in Petri microdishes at a constant temperature and for microscopic observation of said microorganisms, without, however, allowing for simultaneous incubation of biological samples at different temperatures. The description of the patent US 2016/0145562 A1 entitled “A device for monitoring the growth of a biological material” discloses a device for the incubation and optical monitoring of the growth of embryonic cells over time. Accordingly, said invention in its first aspect relates to a device for monitoring the growth of biological material. The device comprises a housing that accommodates two or more separate chambers/compartments containing cell culture plates. Each of the said chambers/compartment comprises a cover, gas inlet and outlet, and heating elements, wherein said device comprises one or more cameras for capturing pictures of the biological material in the culture dish allowing for repositioning. In one embodiment of the first aspect of said invention, preferably all said compartments/chambers comprising cell culture plates comprise a shelf fitted to the culture dish used, said shelf being equipped with a heating element. In one embodiment of the first aspect of said invention, one or more compartments/chambers with culture plates comprises a temperature sensor that allows for precise temperature control in the compartments/chambers with culture plates. The invention allows for simultaneous incubation of microorganisms at different temperatures, without, however, being equipped with sensors providing the control of the current temperature of each incubation chamber. The description of the patent U.S. Pat. No. 8,332,957 B2 entitled “Device and method for the incubation of cells” discloses a device for the incubation of cells and cells in liquid suspensions comprising: sterile or sterilisable mobile container/tank allowing for safe insertion without the risk of contamination of at least one culture dish in which the cells are placed, at least one closed opening for the insertion and/or removal of cells and/or culture medium and/or culture dish and/or from the container and at least one device for generating conditions suitable for culture in the container. According to the invention described, the culture dishes for incubation are transferred to a container/tank and/or are integrated with it. This container protects the culture dishes against external contamination. The term “culture dish” as used herein may relate to culture well plates, culture flasks or other cell carriers suitable for incubation of cell cultures. According to one example, a device for controlling cell culture conditions comprises an incubator with an integrated heating device and a gas feeding device for providing suitable culture conditions in at least one container/tank containing the culture dish. The heating device controls the temperature of the surface on which containers/tanks are placed in which culture dishes are stored. In this case, the culture dish is therefore heated only by the transfer of heat from the heating element to the base of the dish. According to another embodiment, the gas required to obtain suitable cell culture conditions is filtered in a sterile manner and introduced into the container/tank accommodating the culture dish. The gas may be, for example, carbon dioxide CO₂, oxygen O₂, water vapour with the specific temperature required by the culture conditions. Thus, the temperature inside the container/tank is changed by changing the temperature of the feed gas, which can alter, however, the humidity of the air and its condensation in the culture dish, which in turn can lead, for example, to uncontrolled cell growth. The device allows for the incubation of different microorganisms at different temperatures by unidirectional heat transfer from the heating element to the base of the culture dish or by controlling the air temperature. The heating element allows for controlling the temperature of the incubation, but the device does not allow direct measurement of the current temperature of the heating element or the culture dish.

The incubation devices available allow the culture of all the samples present in the incubation chamber at a constant, predetermined temperature. However, different types of microorganisms are characterised by different temperature conditions that mediate the dynamics of their growth and development, which means that during incubation in conventional devices during a single incubation process only cells with the same temperature requirements may be cultured. An example may be provided in the form of an analysis of the growth of bacterial cells and their colonies for characterisation, their identification in order to determine aetiologic causes of infection, inflammation or disease, the accuracy of which depends on the temperature at which the bacterial culture is incubated. To culture microorganisms that need a different (lower or higher) incubation temperature requires the use of an additional device or queuing cell cultures in the same device is necessary. The above inventions describe various devices that allow for the incubation of microorganisms at a stable temperature, atmosphere, isolated from the external environment and in sterile conditions, together with additional functions. If cells could be cultured at different incubation temperatures in the same device and at the same time, it would significantly enhance the functionality of such an incubating device, without, however, having been analysed so far. In addition, in the case of microbiological studies, a large number of field studies are conducted in connection with controlling environmental bacteriological contamination or diagnosing animals that require the use of a mobile, thermally stable, low power incubator to promote the growth of various bacterial preparations for testing. Commonly available incubation devices, due to their size, can be used in laboratory units as stationary devices. This prevents mobile incubation of cells in the field, in medical or veterinary clinics, etc. Surprisingly, the problems outlined above are solved by the invention presented.

The invention relates to a portable incubation device comprising a device housing accommodating an incubation chamber housing defining an incubation chamber with an opening together with a feed system and a temperature control system of the heating module, characterised in that in the incubation chamber there is at least one heating system comprising at least one heating module, preferably two, wherein on the heating system there is a supported a frame supporting a culturing dish, and wherein the temperature control system of the heating module provides continuous real time regulation of the desired incubation temperature in the incubation chamber. Also preferably, the device according to the invention is characterised in that the heating segment is connected to the housing by means of mounting plates. In another preferable embodiment of the device, the main incubation chamber comprises at least one incubation chamber, preferably forming an incubation column. In another, also preferred embodiment of the invention, the device comprises an optical system, more preferably additionally an element controlling the gas composition. More preferably, the device of the invention comprises a light source. Most preferably, the device of the invention is characterised in that it comprises a mechanism for automatically transporting the culture dish collector unit cooperating with the culture dish collector unit. Also preferably, the device of the invention is characterised in that the heating module comprises transducers of electric energy into thermal energy. Most preferably, the device of the invention is characterised in that the spatial configuration of the transducers of electric energy into thermal energy and their number corresponds to the geometry of the culture dish so as to provide an optimum spatial distribution of the heating module temperature.

The device developed allows for the incubation at customised temperature conditions of at least one culture dish without forced air circulation resulting in the drying of the culture substrate at a precisely determined temperature that is measured by a non-contact thermal sensor located on each heating module and controlled by the control system. This allows for the incubation of cell cultures with an accuracy of ±0.2° C. The structure of the device is modular, i.e. it can contain any number of heating segments consisting of two heating modules between which the culture dish is placed, thus allowing for adapting the device as required by the user. Each heating segment allows for incubation in the temperature range from 5° C. above ambient temperature to 44° C. In addition, it is possible to limit the number of heating segments which further optimises and reduces the size of the device, thus allowing for the mobile nature of incubation. The solutions applied allow for optimising the device in terms of energy by reducing energy consumption, reducing power consumption, which for one heating segment is about 1 W, depending on the external ambient temperature. In order to compare the device developed against other commercial incubators, a comparison of sizes and power consumption has been prepared for incubating devices with 10 Petri dishes: the device by Labnet Mini Incubator (http://northamerica.labnetinternational.com/products/mini-incubator) (capacity: ˜9.2 L, power consumption: ˜70 W, incubation accuracy: ±0.5° C.), by Unico Analog Forced Air Incubation (http://www.psscientific.com/shop/incubators/unicoanalogforcedairincubator.aspx) (capacity: ˜6 L, power consumption: ˜80 W, incubation accuracy: ±0.5° C.), developed device (capacity: 4 L, power consumption: ˜11 W, incubation accuracy: ±0.2° C.). The developed device has an intelligent management system of the heating power of each sample. In addition, it also has an integrated automatic transmission system of the culture dish allowing for insertion and removal of the culture dish into/from the incubation chamber without user intervention. In addition, the modular nature of the design allows for the integration thereof with other systems for automated processing, analysis, characterisation and testing of cell cultures in culture dishes.

The term “culture dish” as used herein refers to Petri micro- and macrodishes, circular symmetry multi-well dishes, which allow for culturing microorganisms in liquid and solid media. The device can be used for incubation of prokaryotic and eukaryotic biological cells, but it is dedicated for bacterial and fungal cell cultures. The modular incubation device for customised culture of microorganisms of the invention consists of temperature control systems and automatic transmission of the culture dish, an incubation chamber housing with a covered opening, through which at least one culture dish is inserted, an incubation chamber with at least two heating modules equipped with at least one temperature sensor integrated with at least two mounting plates that are mountable in the incubation chamber, wherein on at least one of which a frame is placed with a culture dish, which is integrated with the automatic transmission system of the culture dish from/to the chamber consisting of a transmission unit of the culture dish and an automatic transmission mechanism of the culture dish collector unit. The incubation of at least one culture dish is implemented by two heating modules, the first of which is placed directly under the frame with the culture dish, and the other is located above the frame with the culture dish. The heating module, which is the source of heat necessary for the development of microorganisms, stabilises the temperature within one slot adapted to accommodate the culture dish.

Each module has a microcontroller designed for reading local temperature values from the sensor, which allows for controlling the heating temperature in real time, and a heater consisting of suitably arranged heating elements. The temperature sensors used allow for continuous regulation of the heating temperature. The heating plate of the heating module comprises an assembly of resistors or other elements allowing for the conversion of electrical energy into thermal one, the arrangement of which on the plate is adapted to the geometry of the culture dish. Preferably, the heating plate consists of transducers of electric energy into thermal energy suitably arranged on the mounting plate. In addition, the distance between the transducers, their number and configuration may vary accordingly to adjust to the conditions of thermal incubation of samples placed in incubation dishes having different spatial geometry. Heating modules on mounting plates mounted in the incubation chamber are mounted in columns (stacked) to form an incubation column. The heating module is equipped with a serial interface for communicating with the other control elements of the device. The culture dish is equipped with an additional thermal sensor allowing for wirelessly controlling the temperature inside the culture dish. The housing of the device consisting of an incubation chamber housing, a bottom and an upper base, it is equipped with a cover made of a heat insulating material and allows for opening or closing the incubation space to which the culture dishes are inserted/removed for the purpose of isolation from the external environment. The housing limits heat losses generated by the heating modules in the incubation chamber, the exchange of heat with the external environment, the exchange of air with the external environment, and, in addition, it provides sterile incubation conditions for microbial cultures. The cover is electro-mechanically controlled so as to automatically open/close the incubation chamber. The cover is equipped with two foldable screens made of ultraviolet radiation-resistant material electronically controlled so as to automatically open/close the incubation chamber. Each of the incubation chambers has its own closure cover that isolates it from environmental effects. The incubation device has an interface to communicate with the master control unit. The device according to the invention is equipped with an electro-mechanical system allowing for automatic insertion/removal of a culture dish in/from the incubation chamber. The transmission unit mechanism of the culture dish designed to place the plate at a user-defined level and place the frame with the culture dish on the heating module carries out these functions in the X, Y and Z axes. The transmission unit mechanism of the culture dish implements movement by means of stepper motors, DC motors and/or servomotors. The device is equipped with a master control unit that controls the temperature of at least one heating module and of the transmission unit mechanism of the culture dish. The incubation system is a partially independent module, so that, in the absence of communication with the main unit, the controller is designed to maintain the incubation regardless of the external conditions, and also in case of power failure the parameters of the samples held are stored in the non-volatile memory for use when power is back, and the incubation process is automatically resumed so as to ensure the highest degree of reliability. For example, an I2C interface is used for the communication between the incubation controller and the heating modules and inside the heating module. The communication system between the elements of the device is a serial interface, which also enables to power all the elements of the device that are connected to it. The device is equipped with an optical system including a detector that are used to control and evaluate the growth and quantity of the microorganisms incubated in the culture dish. Each heating segment is equipped with an optical system including a detector that are used to control and evaluate the growth and quantity of the microorganisms incubated in the culture dish.

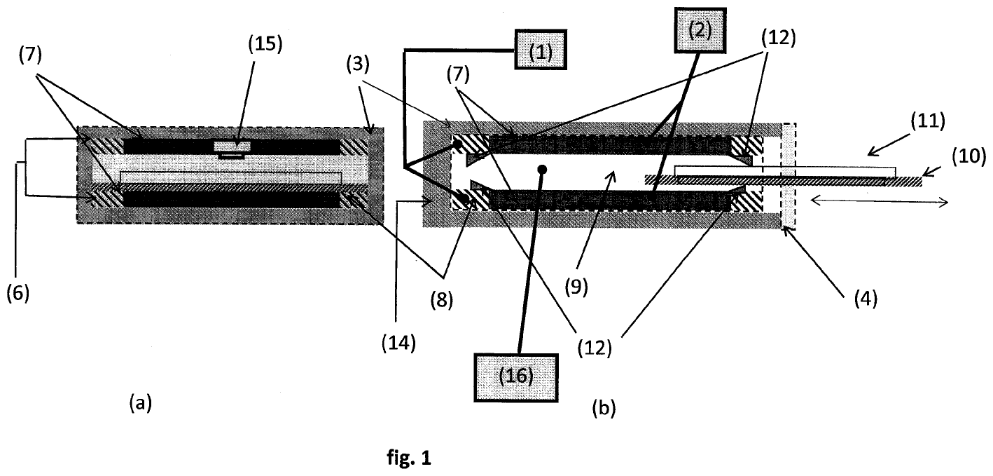

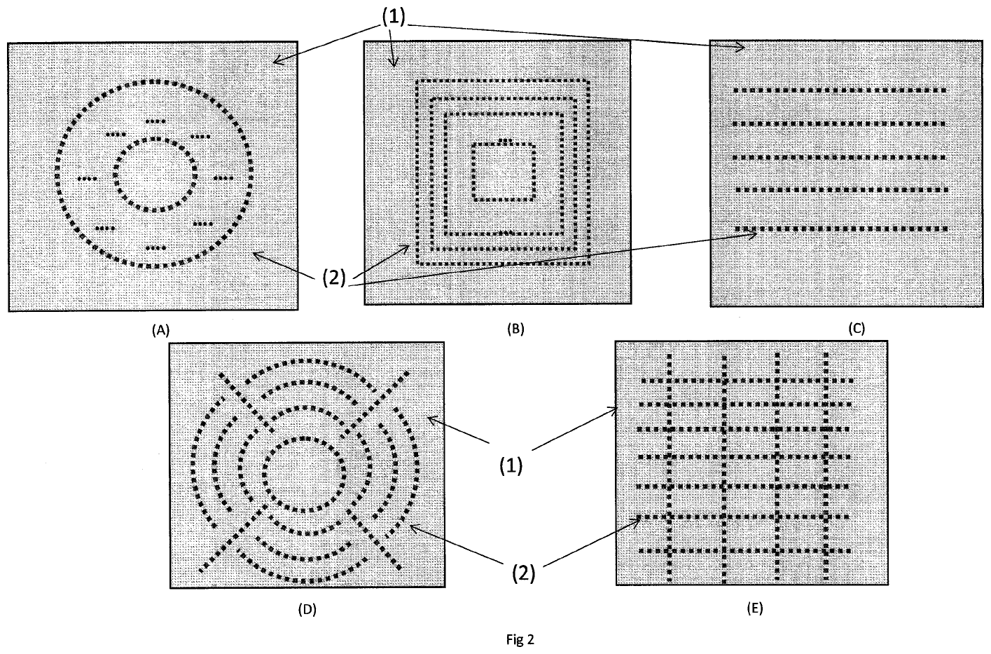

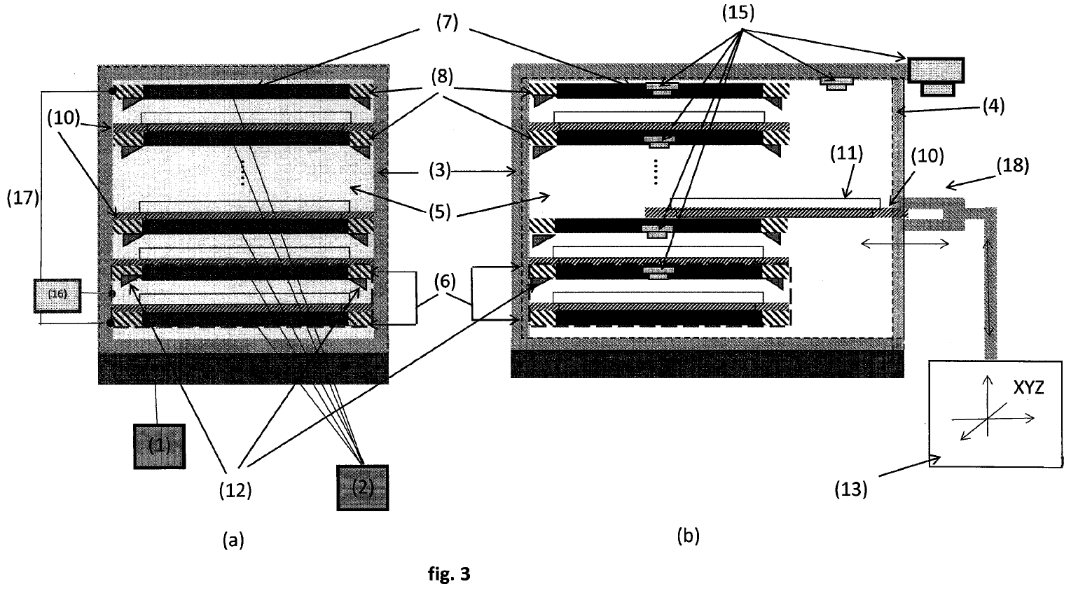

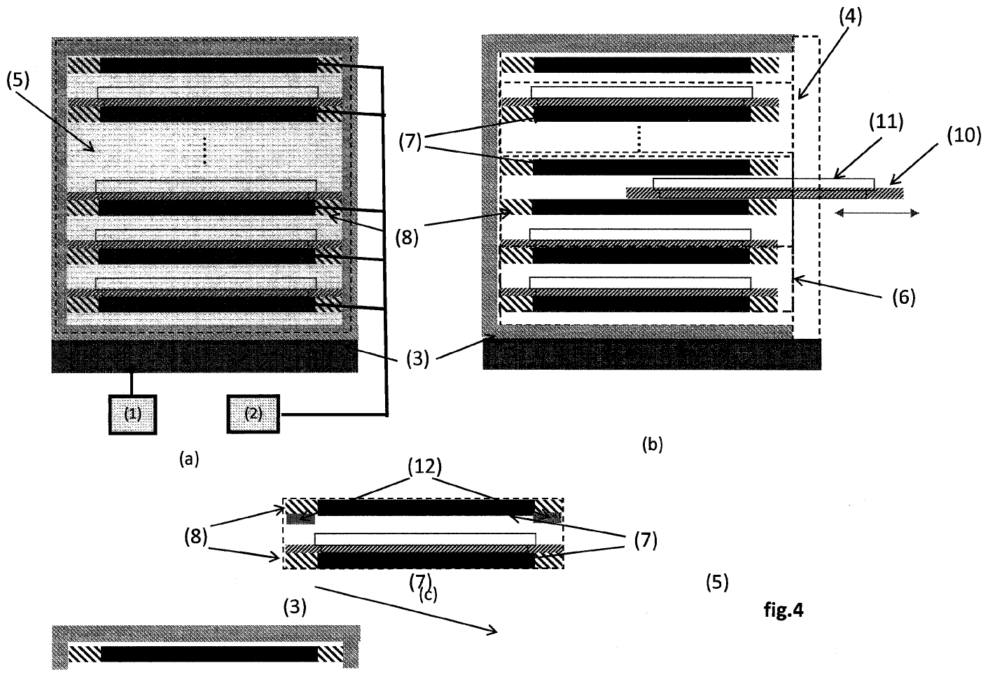

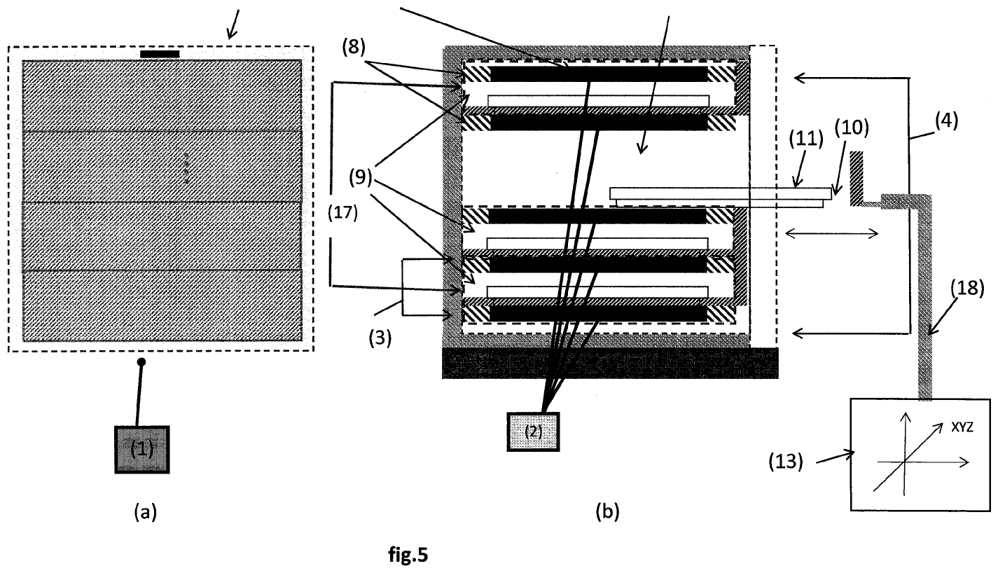

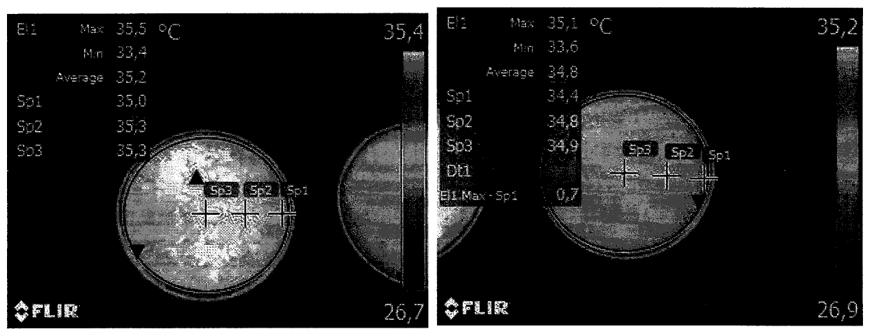

Examples of embodiments of the invention are shown illustrated, where FIG. 1 is a front (a) and side (b) view of a portable incubation device for one culture dish, FIG. 2A, 2B, 2C, 2D, 2E depict examples of spatial configurations of the distribution of transducers of electric energy into thermal energy on the mounting plate, FIG. 3 is a front (a) and side (b) view of an automated modular incubation device for cell culture using multiple dishes, FIG. 4 is a front (a) and side (b) view, and (c) a single heating segment of a stationary incubation device for multiple culture dishes; and FIG. 5 is a front (a) and side (b) view of a stationary incubation device for multiple culture dishes having separate incubation chambers; and FIG. 6 depicts the optimisation of the incubation process by achieving a stable, even distribution of the temperature of the culture substrate placed in a Petri dish (symbols Sp1, Sp2, Sp3 refer to local temperatures of the culture substrate).

### Example 1: Portable Incubation Device for a Single Culture Dish

According to a first aspect of the invention, a portable incubation device for customised culture of microorganisms has been developed in order to provide a portable incubator for a single culture dish. The invention according to the first embodiment is illustrated in FIG. 1, where (a) is a front view, and (b) is a side view. The portable incubation device for customised culture of microorganisms comprises an incubation chamber housing (3) accommodating an integrated feed system (1) and an integrated temperature control system of the heating module (2), wherein the incubation chamber housing (3) has a covered opening (4) through which the culture dish (11) is inserted. One heating segment (6) consists of two heating modules (7), equipped with at least one temperature sensor, integrated with at least two mounting plates (8) allowing them to be mounted in an incubation chamber (9), at least on one of which a frame (10) is placed with a culture dish (11), and the transfer of heat necessary to obtain the suitable thermal conditions is carried out from the top and bottom of the culture dish located between the two heating modules, so that the portable incubation device allows for mobile incubation of a single culture dish at a selected temperature, that is to say in customised thermal conditions.

The heating module (7) is equipped with an additional thermal sensor allowing for wirelessly controlling the temperature inside the culture dish. The housing of the device (14) consists of an incubation chamber housing (3), a bottom and an upper base, it is equipped with a cover made of a heat insulating material and allows the user to open or close the incubation space to which the culture dishes are inserted/removed for the purpose of isolation from the external environment. The device is equipped with additional light sources (12) in the form of LEDs emitting radiation in the range from 400 to 1100 nm, which allow for illuminating microorganisms during incubation or in the form of a light source emitting UVC-UVB radiation, which is activated before or after incubation in order to sterilise the inside of the incubation chamber. The device is equipped with an additional optical system (15) consisting of a camera and a lens for controlling the growth of microorganisms and their characterisation. The device is equipped with additional elements controlling the gas composition (16) of the atmosphere inside the incubation chamber. The heating plate of the heating element (7) is coated with a thermal conductive material allowing for even temperature distribution. The mounting plate (8) of the heating module and the frame for the culture dish (10) are made of a heat insulating material.

### Example 2: Embodiment of the Heating Module

Each heating module (see FIG. 2) consists of heating elements (2) suitably arranged on the mounting plate (1). The temperature sensors used allow for continuous control and regulation of the heating temperature. The heating plate of the heating module comprises an assembly of resistors or other elements allowing for the conversion of electrical energy into thermal one, the arrangement of which on the plate is adapted to the geometry of the culture dish. Preferably, the heating plate consists of transducers of electric energy into thermal energy suitably arranged on the mounting plate. FIGS. 2A, 2B, 2C, 2D, 2E illustrate examples of spatial configurations of the distribution of transducers of electric energy into thermal energy on the mounting plate. In addition, the distance between the transducers, their number and configuration may vary to adjust to the conditions of thermal incubation of samples placed in incubation dishes having different spatial geometry. The suitable spatial configuration of the transducers, their number and a suitable heating temperature control system allow for the most optimal real-time adjustment of the surface temperature distribution of the heating module allowing for a far more functional incubation of samples placed in culture dishes having different spatial geometry.

### Example 3: Automated Modular Incubation Device for Cell Culture using Multiple Dishes

According to a second embodiment of the invention, the modular design allowed for obtaining a a portable incubation device for personalised culture of microorganisms in the form of an incubation column for culturing cells in more than one culture dish with cells at an individually selected temperature, which operates in a fully automated manner, reduces power consumption and allows for the integration thereof with other devices, functional modules, e.g. for automatic culture plating and preparation of microorganisms samples, characterisation of their properties: growth dynamics, phenotypic characteristics, morphology, optical properties or recording additional optical patterns of microorganisms, etc. The invention according to the second embodiment is shown in FIG. 3.

A modular incubation device for customised culture of microorganisms according to the second embodiment of the invention comprises an integrated feed system (1), integrated systems controlling the temperature of the heating segments (2) and an automatic transmission system of the culture dish collector unit (18), an incubation chamber housing (3) with a covered opening (4), through which at least one culture dish is inserted, a main incubation chamber (5) divided into more than one separate smaller chamber allowing for the incubation of samples at different temperatures, each of which consists of a heating segment (6) containing two heating modules (7) placed on mounting plates (8) between which a frame (10) is inserted with the culture dish (11), integrated with an automatic transmission system consisting of the culture dish collector unit (18) and an automatic transmission mechanism of the culture dish collector unit (13), which allows the culture dish to be transmitted from/to each heating segment by ejecting and displacing the culture dish alone or the culture dish together with the frame outside of the main incubation chamber. Each heating segment allows for obtaining individual temperature conditions for incubating the cell culture, with the transfer of heat to the culture dish implemented by two heating modules located above and below the culture dish, insertion and removal of the culture dish is implemented automatically, and the entry opening (4) to the incubation chamber is covered with an automatically opening/closing cover/door/screen.

It is preferable that introducing the culture dish into one of the heating segments does not lead to a change in the temperature conditions of the incubation of the remaining culture dishs in other heating segments as is the case in conventional embodiments where various culture dishs are incubated in a common incubation chamber.

The housing of the device (14) comprises more than one heating segment (6) and provides thermal insulation from the external environment. The housing opening (4) limits the incubation space to be separated from the outside environment by means of a fully automatic and fully integrated lid/door/dome that integrates with the control system. Each heating segment (6) enables the culture of microorganisms in a culture dish at various temperatures adapted to the specific requirements of microorganism culture in the incubation device. Each heating segment (6) is thermally insulated from neighboring heating segments (6) by means of a thermal barrier that restricts the flow of heat and air between the segments. Each heating module (7) generates heat energy with a uniform temperature distribution adapted to the geometry of the culture dish. All heating segments (6) are stacked one on top of another to form an incubation column (17) in the incubation space. Transport of the culture dish to/from the heating segment (6) takes place automatically. The incubation device for individualized culture of microorganisms is integrated with other functional modules such as automatic seed sampling, sample preparation, characterization of samples, which ensures a fully automated culture and characterization of cell culture properties such as growth dynamics, colony size, phenotypic characteristics, morphology, optical or optical pattern registration of these farms. The automatic transport unit of the dishwasher assembly (13) to/from the heating segment (6) is integrated with additional sample preparation units, their testing or characterization allowing fully automated transport of the culture dish between successive heating segments (6) and additional functional modules. The control unit also controls the operation of additional functional modules such as that of sample preparation. The device is equipped with an additional optical system (15) consisting of a camera including a lens that allows for recording pictures of the cell culture during and after incubation in order to characterise the phenotypic characteristics of such culture. Each heating segment (6) comprises light sources (12) in the form of LEDs emitting radiation from the spectral range of 400 to 1100 nm, which illuminate the culture dish during incubation in order to stimulate the dynamics of the growth of microorganisms. Each heating segment (6) is equipped with an additional element controlling the gas composition (16) inside the segment in order to provide optimum conditions for the growth of microorganisms. The device is equipped with an intelligent, automated power control system for each heating segment (6). The device consists of more than one incubation column located next to each other, each of which comprises at least one heating segment (6) with a culture dish (11) operated by at least one automatic transmission mechanism of the culture dish unit (13) in the heating segment (6) of each of the incubation columns. The device consists of more than one incubation column located opposite to each other, each of which comprises at least one heating segment (6) with a culture dish, operated by at least one automatic transmission mechanism of the culture dish unit (13) in the heating segment (6) of each of the incubation columns.

### Example 4: Stationary Incubation Device for Multiple Culture Dishes

According to the third embodiment of the invention, it is possible to develop a stationary incubation device for thermally customised culture of microorganisms in multiple culture dishes that reduces power consumption. The invention according to the third embodiment is shown in FIG. 4.

A modular incubation device for customised culture of microorganisms according to the third embodiment of the invention comprises a feed system (1), systems controlling the temperature of the heating segments (2) an incubation chamber housing (3) with a covered opening (4), through which more than one culture dish is inserted, a main incubation chamber (5) divided into more than one separate smaller chamber allowing for the incubation of samples at different temperatures, each of which consists of a heating segment (6) containing two heating modules (7) placed on mounting plates (8) between which a frame (10) is inserted with the culture dish (11). The user autonomously places the culture dish (11) in each of the heating segments, opens/closes the door/screen/cover covering the entry opening (4) to the main incubation chamber (5), and the device reduces the power consumption and allows for incubating the culture dish (11) with microorganisms in customised thermal conditions. The housing of the device (14) comprises more than one heating segment (6) and provides thermal insulation from the external environment. Each heating segment (6) comprises a light source (12) in the form of LEDs emitting radiation from the spectral range of 400 to 1100 nm, which illuminate the culture dish (11) during incubation in order to stimulate the dynamics of the growth of microorganisms. Each heating segment (6) enables the culture of microorganisms in a culture dish at various temperatures adapted to the specific culture requirements in the incubation device. All heating segments (6) are stacked one on top of another to form an incubation column in the incubation space. The device is equipped with an intelligent, automated power control system for each heating segment (6).

### Example 5: Stationary Incubation Device for Multiple Culture Dishes Having Separate Incubation Chambers

According to the fourth embodiment of the invention, it is possible to develop a stationary incubation device for customised culture of microorganisms in multiple culture dishes that reduces power consumption. The invention according to the fourth embodiment is shown in FIG. 5.

A modular incubation device for customised culture of microorganisms according to the fourth embodiment of the invention comprises systems controlling the temperature of the heating module (2), incubation chamber housing (3) of the incubation column (17) comprising at least heating segment (6) delimiting the main incubation chamber (5) with an opening (4) through which more one culture dish (11) is inserted, the main incubation chamber (5) divided into incubation chambers(9) allowing for the incubation of samples at different temperatures, each of which consists of a heating segment (6) containing two heating modules (7) placed on mounting plates (8) between which a frame (10) is inserted with the culture dish (11). The frame (10) on which the culture dish (11) is placed, upon insertion into the incubation chamber (9) covers the entry opening (4) through which it has been inserted for the purpose of separation from the external environment. The device is equipped with frames (10) transmitting the culture dish (11) allowing the covering of the entry opening (4) of the incubator for the purpose of separation from the external environment. The housing of the incubation chamber (3) comprises more than one heating segment (6) and provides thermal insulation from the external environment. Each heating segment (6) comprises light sources (12) in the form of LEDs emitting radiation from the spectral range of 400 to 1100 nm, which illuminate the culture dish (11) during incubation in order to stimulate the dynamics of the growth of microorganisms. Each heating segment (6) enables the culture of microorganisms in a culture dish (11) at various temperatures adapted to the specific culture requirements in the incubation device. All heating segments (6) are stacked one on top of another to form an incubation column (17) in the incubation space. The device is equipped with an automated mechanism allowing for the insertion and removal of the frame with the incubation dish from the incubation chamber and transfer to a deposition site. The device is equipped with an intelligent, automated power control system for each heating segment. The device consists of multiple incubation columns (17) operated by a single automatic transmission mechanism of the culture dish collector unit (13) and the the culture dish collector unit (18).

1. Portable incubation device comprising a device housing 14 accommodating an incubation chamber housing 3 defining an incubation chamber 9 with an opening 4 together with a feed system 1 and a temperature control system of the heating module 2, characterised in that in the incubation chamber 9 there is at least one heating system 6 comprising at least one heating module 7, preferably two, wherein on the heating system 6 there is a supported a frame 10 supporting a culturing dish 11, and wherein the temperature control system of the heating module 2 provides continuous real time regulation of the desired incubation temperature in the incubation chamber 9.

2. A device according to claim 1, characterised in that the heating segment is connected to the housing by means of mounting plates 8.

3. A device according to claim 1 or 2, characterised in that the main incubation chamber 5 comprises at least one incubation chamber 9, preferably forming an incubation column 17.

4. A device according to any of the claims 1 to 3, characterised in that it comprises an optical system 15, more preferably a gas composition control element 16.

5. A device according to any of the claims 1 to 4, characterised in that it comprises a light source 12.

6. A device according to any of the claims 1 to 5, characterised in that it comprises a mechanism for automatically transporting the culture dish collector unit 13 cooperating with the culture dish collector unit 18.

7. A device according to any one of claims 1 to 6, characterised in that the heating module comprises transducers of electric energy into thermal energy.

8. A device according to claim 7, characterised in that the spatial configuration of the transducers of electric energy into thermal energy and their number corresponds to the geometry of the culture dish so as to provide an optimum spatial distribution of the heating module temperature.
